(19) United States
(12) Patent Application Publication
(10) Pub. No.: US 2007/0141641 A1
(43) Pub. Date: Jun. 21, 2007

(54) Compositions & methods for reformulating biological membranes for arrays

(76) Inventors: Ye Fang, Painted Post, NY (US); Ann M. Ferrie, Painted Post, NY (US)

Correspondence Address: CORNING INCORPORATED, SP-TI-3-1, CORNING, NY, 14831, US

(21) Appl. No.: 11/699,650

(22) Filed: Jan. 30, 2007

Related U.S. Application Data
(62) Division of application No. 10/651,554, filed on Aug. 29, 2003.

(51) Int. Cl. G01N 33/567; G01N 33/53

(52) U.S. Cl. 435/7.2

(57) ABSTRACT

A standardized, buffered solution or “ink” composition for re-formulating or suspending biological membranes used in the fabrication of membrane arrays. The composition can enhance assay performance and prolong the shelf life of biological membrane arrays. The ink composition comprises a combination of at least two of the following six classes of reagents: 1) a pH buffer reagent; 2) a monovalent or divalent, inorganic salt; 3) a membrane stabilizer; 4) a solution viscosity control reagent; 5) a water-soluble protein; or, 6) a protease inhibitor. A method for fabricating a membrane array using the present ink composition is also described.

### FIELD OF INVENTION

The invention relates to biological membrane arrays, particularly membrane-protein arrays, which may be used to perform biological, biochemical or chemical binding assays on a solid surface. In particular, the invention pertains to a buffered solution or “ink” composition for re-formulating or suspending biological membranes employed in the fabrication of such arrays. The invention also describes a method to enhance assay performance and prolong the shelf life of biological membrane arrays.

### BACKGROUND

In recent years, the biological, clinical, and pharmaceutical research communities have turned to biological membrane or membrane-protein arrays as valued research tools. Biological or cellular membranes are selective barriers that separate individual cells and cellular compartments. Cell membranes play extremely important roles in maintaining the integrity of living cells. Membranes regulate the transport of molecules, contain molecules responsible for cell adhesion in the formation of tissues, control information flow between cells, generate signals to alter cell behavior, as well as can separate charged molecules for cell signaling and energy generation through molecules embedded in or associated with cellular lipid membranes. Given this importance of cell membranes, and since a large fraction of drug targets are membrane bound proteins, biological membrane arrays, in particular membrane-protein arrays, would greatly benefit researchers' understanding of the structure and function of cell membranes and membrane-associated molecules, as well as accelerate the drug-discovery process for identifying potential drugs that target cell membrane proteins¹ (e.g., G-protein coupled receptors, ion channels, tyrosine kinases, etc) and other important processes involving cell membranes (e.g., toxin and virus recognition and infection).
¹ For instance, G-protein coupled receptors (GPCRs) represent the single most important class of drug targets—approximately 50% of current drugs target GPCRs; about 20% of the top 50 best selling drugs target GPCRs; more than $23.5 billion in pharmaceutical sales annually are ascribed to medications that address this target class. (Drews, J., “Drug Discovery: A Historical Perspective” Science 2000, 287, 1960-1963; Ma, P., and Zemmel, R., “Value of Novelty” Nat. Rev. Drug Discov. 2002, v. 1, 571-572.)

Cell membranes are assemblies of membrane-proteins, carbohydrates, and lipids held together by non-covalent forces. Membrane proteins determine the functionality of cell membranes, serving as pumps, gates, receptors, cell adhesion molecules, energy transducers, and enzymes. Peripheral membrane proteins are associated with the surfaces of membranes, while integral membrane proteins are embedded in the membrane and may pass through the lipid bilayer one or more times. All integral proteins bind asymmetrically to the lipid bilayer. Asymmetry of membrane proteins is established during their biosynthesis and maintained throughout the proteins lifetime. Each type of integral membrane protein has a single, specific orientation with respect to the cytosolic and exoplasmic faces of a cellular membrane. All molecules of any particular integral membrane protein, such as glycphorin, lie in the same direction. This absolute asymmetry in protein orientation generates the different characteristics associated with the two faces of a membrane. A special set of proteins are cell junctions, which anchor cells together (desmosomes), occlude water passing between cells (tight junctions), and allow cell to cell direct communication (gap junctions).

Another cell membrane constituent includes carbohydrates covalently linked to proteins (glycoproteins) or lipids (glycolipids). The carbohydrate groups provide part of the structure that enables the glycolipid and glycoprotein molecules to perform recognition, reception and adhesion functions. For instance in a plasma membrane, all of the O— and N-lined oligosacharides in glycoproteins and all of the oligosaccharides in glycolipids are on the exoplasmic surface. In the endoplasmic reticulum, they are found on the interior, or lumenal, membrane surface.

A lipid bilayer, with hydrophobic cores made up predominately of fatty acid chains and hydrophilic outer surfaces, constitutes the major structural element of the lipid portion of a cell membrane. Typical lipids within cell membranes include phospholipids composed of glycerol, fatty acids, phosphate, and a hydrophobic organic derivative such as choline or phosphoinositol. The lipids of membranes create a hydrophobic barrier between aqueous compartments of a cell. In both pure phospholipid bilayers and in natural membranes, thermal motion permits phospholipid and gycolipid molecules to rotate freely around their long axes and to diffuse laterally within the membrane leaflet. Because such movements are either lateral or rotational, the fatty acyl chains remain in the hydrophobic interior of the membrane. Cholesterol and its derivatives constitute another important class of membrane lipids, the steroids. Cholesterol regulates membrane fluidity and is a part of membrane signaling systems.

Bilayer-lipid membranes adsorbed on solid supports, also known as “supported bilayer-lipid membranes,” can mimic the structural and functional roles of biological membranes. Supported membrane structures can be useful in various applications, such as binding and other studies aiming at gaining a better understanding of the biological membrane. (See Sackmann, E. Science 1996, 271, 43-48; Bieri, C. et al., Nature Biotech., 1999, 17, 1105-1108; Groves, J. T. et al., Science 1997, 275, 651-653; Lang, H. et al., Langmuir 1994, 10, 197-210; Plant, A. L. et al., Langmuir 1999, 15, 5128-5135; and Raguse, B. et al., Langmuir 1998, 14, 648-659.) For example, biosensors based on supported lipid membranes have found a number of applications including toxin detection (Puu, G. “An Approach for Analysis of Protein Toxins Based on Thin Films of Lipid Mixtures in an Optical Biosensor”, Anal. Chem. 2001, 73, 72-79).

Fabrication of biological membrane arrays, however, is a relatively challenging process for several reasons, including the difficulties associated with preserving the correctly-folded conformation of proteins in an immobilized state. For example, since G-protein coupled receptors (GPCRs) require an association of a lipid membrane to retain their correct, folded conformations and functions, the stability and integrity of the lipid membrane is important. When creating arrays of such membrane-proteins on a substrate, the lipid membranes need to be elevated or offset from the substrate surface in order to avoid misfolding or dysfunction of the extra-membrane domains of the protein or receptor molecules. Physical contact with the substrate surface can induced such dysfunctions in the receptor. Furthermore, to produce a robust bioassay, the receptor lipid membranes that are deposited need to be stably associated with the surface. Covalent immobilization of the entire membrane, however, is not desirable since lateral mobility is an intrinsic and physiologically important property of biological membranes; a feature one would like to maintain. For instance, GPCR-G protein complexes should be preserved after being arrayed onto a surface since the correct configuration of the receptor and G protein is a prerequisite for the binding of agonists to the receptor with physiological binding affinity.

The advent of DNA microarray technology has developed several printing technologies that are amenable to commercial production-scale fabrication. These printing technologies generally involve printing and post-printing processes under ambient conditions. Adaptation of these technologies would greatly facilitate the fabrication of membrane microarrays. Membrane microarrays can be fabricated in two fundamentally different ways. The first approach directly immobilizes membranes onto a micropatterned substrate having membrane-binding and non-binding regions (Boxer, S. G. et al. Science 1997, 275, 651-653; and Boxer, S. G. et al. Langmuir 1998, 14, 3347-3350). Extending this approach to fabricate microspots of different compositions is challenging because of certain registration issues. The second approach directly prints solutions of membranes or membrane proteins onto a membrane-binding surface. For example, micro-pipetting techniques have been used to spatially address each lipid-binding region (Cremer, P. S. et al., J. Am. Chem. Soc. 1999, 121, 8130-8131). The printing of biological membranes, however, are done on substrates immersed in buffer because of concerns with membrane stability upon exposure to air. It is known that lipid membranes are susceptible to changes in the environment. Lipid membranes supported on solid substrates are especially susceptible to desorption and/or deterioration in structure when withdrawn through an air-water interface. These issues have hampered the development of membrane microarrays.

The deficiency of biological membrane microarrays having good quality and functional membrane-proteins has been viewed a major gap in the development of protein microarray technologies (Mitchell, P., “A Perspective on Protein Microarrays,” Nat. Biotechnol. 2002, v. 20, 225-229). Therefore, methods to reduce the risk associated with the deterioration of lipid membranes when exposed to ambient conditions are needed in order to improve the printing and array quality of biological membrane microarrays.

### SUMMARY OF THE INVENTION

The present invention provides, in part, an ink or medium for reformulating biological membranes, which may be deposited on a solid support. The medium has a composition that comprises a combination of at least two of the following classes of reagents: 1) a pH buffer reagent having a desired pH from about 5.8 to about 7.8; 2) a monovalent or divalent, inorganic salt at a concentration of about 1 mM to about 500 mM; 3) a membrane stabilizer at a concentration of about 1% to about 25% or 30% (wt.); 4) a solution viscosity control reagent at a concentration of about 1% to about 55% (vol.); 5) a water-soluble protein at a concentration of about 0.01% to 3% (wt.); and, 6) a protease inhibitor at a concentration of about 0.01 mM to about 100 mM. The standardized, buffered medium or composition can be used to fabricate biological membrane arrays that exhibit high quality and desired properties (e.g., biological membrane microspot with low autofluorescence, ideal morphology, limited spreading of biological membranes after printing, target binding with high specificity, optimized binding signals, longer shelf life of printed arrays, and less stringent storage conditions). Biological membranes in a microarray may take the form of either a supported lipid bilayer membrane, or a bilayer vesicle, or a lipid micelle, or at least a partially free-suspended lipid membrane, or a lipid membrane in a nano-channel of a substrate (e.g., nano-porous slide or plate), with or without embedded membrane-proteins (e.g. GPCR, ion channel).

In another aspect, the present invention pertains to a method for making a biological membrane array. Given the difficulties associated with fabrication of biological membrane arrays, the present invention can reduce the risks associated with the deterioration of lipid membranes, and improve the stability, quality, and assay performance of such arrays.

The method comprises contacting or otherwise depositing on a solid support with an ink solution according to the present invention. Depositing step further comprises immersing a tip of a pin into the medium; removing said tip from the medium with the medium adhered to the pin tip; and transferring the ink solution to the solid support. The depositing step can be repeated a plurality of times to provide one or more arrays of biological membranes. This can be accomplished, for example, by using a typographic pin printing.

Additional features and advantageous of the present invention will be revealed in the following detailed description. Both the foregoing summary and the following detailed description and examples are merely representative of the invention, and are intended to provide an overview for understanding the invention as claimed.

### BRIEF DESCRIPTION OF THE FIGURES

FIG. 1A and FIG. 1B are false-color fluorescence images of two membrane microarrays made using six different GPCR-membrane preparations (from left to right, they are HEK cell membrane as control, muscarinic receptor subtype 1 (M1), motilin receptor (MOTR), neurotensin receptor subtype 1 (NTR1), opioid receptor like subtype 1 (ORL1) and delta 2 opioid receptor (OP1)).

FIG. 2 is a false-color fluorescence image in Cy3 channel of a microarray with motilin receptors (MOTR) after incubation with an assay buffer alone.

FIGS. 3A and 3B are false-color fluorescence images in Cy3 channel of motilin receptor (MOTR) microarrays after incubation with an assay buffer containing ˜4 nM Bodipy-TMR-motilin 1-16 (BT-MOT) in either the absence (A, total) or presence (B, non-specific) of ˜2 μM motilin 1-16 (MOT).

FIGS. 4A and 4B are graphical representations of the binding of target to a probe receptor. Graph 4A shows relative fluorescence (RFU) of total (white bar) and non-specific (black bar) binding for each of the columns in FIGS. 3A and 3B. Graph 4B shows the percentage of inhibition of BT-motilin binding to the microarrays of MOTR obtained from different suspensions in the columns of FIGS. 3A and 3B.

FIGS. 5A and 5B are plots of the fluorescence intensity due to specific binding of BT-motilin 1-16 to the MOTR microarrays, as a function of concentration of BT-motilin 1-16.

FIG. 6A is a schematic representation of a microspot having biological membranes surrounded by bovine serum albumin (BSA). The biological membranes are suspended in a buffered solution containing BSA and used for deposition onto a surface to form a microspot.

The BSA molecules tend to form a closely packed layer around the biological membranes confining them within a defined area of the microspot. The BSA limits the ability of the membranes to spread during printing and post-printing process, and stabilizes the membranes. Occasionally, the BSA molecules may fill-in defect areas of the membrane microspot.

FIG. 6B is a false-color fluorescence image in Cy3 channel of a microarrays containing δ2-opioid receptor. The δ2-opioid receptor membrane preparation is re-suspended with a buffered solution containing 0.5% Cy3-labeled BSA. The microarray is reacted with a binding solution containing 4 nM Cy5-Naltrexone. The image shows that the signal at the center of each microspot is relatively lower than the signal of the BSA molecules surrounding the receptor microspots.

FIG. 6C is a false-color fluorescence image in Cy5 channel of FIG. 6B. The image shows Cy5-Naltrexone binding in the defined area of the microspots, but not outside.

FIG. 7 is a histogram presenting data comparing the assay performance of β1 arrays in the presence of different concentrations of trehalose. A first β1 array is assayed with 2 nM BT-CGP12177 (white bar). Another β1 array is assayed with 2 nM BT-CGP 12177 in the presence of 2 μM CGP 12177 (black bar). The results show clearly that the presence of trehalose improves significantly assay performance in terms of its sensitivity without compromising the ligand binding specificity of the arrays. In a number of buffer samples containing different amounts of trehalose, β1 membrane preparations are suspended and used to fabricate a β1 array on a single GAPS-coated slide. After printing and incubation in a humidity chamber, the slides are dried under vacuum for two hours.

FIGS. 8A & 8B present data comparing the assay performance of β1 arrays in the presence of different concentration of trehalose after being stored at room temperature under vacuum for about seven days. FIG. 8A is a histogram representing the binding signal observed after a β1 array is assayed using 2 nM BT-CGP12177 (white bar); and another β1 array assayed using 2 nM BT-CGP 12177 in the presence of 2 μM CGP 12177 (black bar). FIG. 8B is a graph that shows the percentage of binding of BT-CGP to the β1 arrays is inhibited by CGP 12177. Results indicate that after storage for about one week under less stringent conditions than usual, the arrays of beta1 treated with trehalose preserved their biological activity very well, while β1 arrays in the absence of trehalose lost a great extent of their biological functionality.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

The term “cocktail solution” refers to a medium (e.g., buffered or aqueous solution) having a mixture either of different labeled ligands or of different compounds. Alternatively, in some embodiments, a mixture of both ligands and compounds may be present together in solution.

The term “cognate,” “corresponding,” or “paired” refers to the reciprocal moiety of a molecule to another; in particular, a ligand that can bind specifically to a given receptor is called a ligand-receptor pair.

The term “compound,” “target,” or “target compound” as used herein refers to a biological molecule, biochemical or chemical entity, molecule, or pharmaceutical drug candidate to be detected.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon. The surface may have an amine-presenting functionality (e.g., γ-amino-propylsilane (GAPS) coating), or may be coated with amine presenting polymers such as chitosan and poly(ethyleneimine).

The term “GPCR membrane” or “GPCR membrane fragment” refers to a biological membrane or cell membrane fragment having a GPCR embedded within a membrane layer, or a micelle having a GPCR reconstituted within the micelle.

The term “ink,” “medium,” or “composition” refers to a buffered medium or aqueous solution containing components or reagents that can stabilize a biological membrane either in solution or after deposition onto a substrate, and/or improve the consistency or reproducibility of the amount of membrane-containing solution transferred from a disposition device to the substrate. The components include a combination of six classes of reagents: 1) a pH buffer reagent; 2) a monovalent or divalent, inorganic salt; 3) a membrane stabilizer; 4) a solution viscosity control reagent; 5) a water-soluble protein; and/or, 6) a protease inhibitor. In some embodiments, a mixture of at least two of the six classes of components with biological membranes may be present together in solution.

The term “microspot” refers to a discrete or defined area, locus, or spot on the surface of a substrate, containing a biological or chemical probe. The term “receptor microspot” refers to a microspot containing a deposit of biological membrane presenting binding functional moieties or molecules, such a ganglioside, phosphatidylinositol phosphate (PIP), sphingolipid, or membrane-proteins. The membrane-protein may include a GPCR, a ligand-gated ion channel receptor, a tyrosine kinase receptor, serine/threonine kinase receptor, or guanylate cyclase receptor.

The term “probe” or “probe receptor” refers to a receptor molecule (e.g., GPCR), which according to the nomenclature recommended by B. Phimister (Nature Genetics 1999, 21 supplement, pp. 1-60.), is immobilized to a substrate surface. Preferably, probes are arranged in a spatially addressable manner to form an array of microspots. When the array is exposed to a sample of interest, molecules in the sample selectively and specifically binds to their binding partners (i.e., probes). The binding of a “target” to the probes occurs to an extent determined by the concentration of that “target” molecule and its affinity for a particular probe.

The term “substrate” or “substrate surface” as used herein refers to a solid or semi-solid, or porous material (e.g., micro- or nano-scale pores), which can form a stable support. The substrate surface can be selected from a variety of materials.

### Section II—Detailed Description

Previously, we have demonstrated that one may fabricate GPCR microarrays using conventional robotic pin printing technologies and cell membrane preparations containing GPCRs from a cell line over-expressing the receptor. (U.S. patent application Ser. No. 09/974,415 (U.S. Patent Publication No. 2002/0019015 A1), and Ser. No. 09/854,786, (U.S. Patent Publication No. 2002/0094544 A1) the contents of which are incorporated herein by reference). Also, we have described certain methods for fabricating biological membrane microarrays on substrates presenting certain surface chemistries. (U.S. patent application Ser. No. 10/300954, incorporated herein by reference.) These kinds of arrays can be prepared under ambient conditions, stored at about 4° C., and still retain their functionality for an extended period of time thereafter.

For biological membrane microarrays fabricated using cell membrane preparations containing GPCRs from a cell line over-expressing the receptor, a number of specifications relating to the GPCR membrane preparations have been found to play important roles in array printing quality, assay sensitivity and robustness. For example, the particular cell line used and the concentration of the active receptor (Bₘₐₓ in pmol/mg of total protein) can determine the assay sensitivity, while the total protein concentration, the homogeneity and buffer composition of membrane preparations can affect in a significant way the printing quality and array performance. Membrane preparation homogeneity influences the packing density and uniformity of membrane fragments within a microspot, as well as the reproducibility of printing; smaller and more homogeneous membrane fragments yield membrane microspots with better packing density and uniformity, and result in improved printing reproducibility.

Moreover, a buffer composition, in which the biological membranes are suspended, plays a role in printing quality and array performance. Not only can a buffer composition affect the functionality of the membrane proteins, but the buffer also affects the wetting and de-wetting properties of the deposition device used. This factor is important in when one endeavors to produce arrays of high quality. Commercially available GPCR membrane preparations are suspended typically in variable, non-standardized buffer compositions, which have a wide range for the values of Bₘₐₓ and total concentration of membrane-proteins within the preparation. When commercially available GPCR membranes are used directly to make a GPCR array, the resulting array does not perform as well as desired in terms of its properties, such as microspot morphology, stability, and/or ligand-binding specificity. Hence, GPCR membrane preparations should be reformulated so as to achieve better printing reproducibility and better array quality.

In response and to overcome these issues relating to high-quality arrays, the present invention provides an ink or medium composition and methods to reformulate biological membrane preparations for fabricating micrarrays. The microarrays that make use of the present method have improved printing quality (i.e., consistent microspot morphology and reproducibility), prolonged storage shelf-life, and assay performance (i.e., sensitivity and robustness). The method for improving the fabrication and use of the biological membrane array comprises: providing an ink solution, as described herein, reformulating a biological membrane preparation using the ink solution, and depositing the reformulated biological membrane preparation(s) onto a substrate surface to form a microarray. The reformulating step comprises the further steps of: mixing the ink solution with the biological membrane preparation and homogenizing the preparation.

A. Ink Compositions for Reformulating Biological Membranes

The present invention provides, in part, an ink or medium for reformulating a solution of biological membranes, which may be deposited on a solid support. The medium has a composition that comprises a combination of at least two of the following classes of reagents: 1) a pH buffer reagent having a desired pH from about 5.8 to about 7.8; 2) a monovalent or divalent, inorganic salt at a concentration of about 1 mM to about 500 mM; 3) a membrane stabilizer at a concentration of about 1% to about 27% (wt.); 4) a solution viscosity control reagent at a concentration of about 1% to about 55% (vol.); 5) a water-soluble protein at a concentration of about 0.01% to 3% (wt.); and, 6) a protease inhibitor at a concentration of about 0.01 mM to about 100 mM.

Preferably, the medium has a pH from about 6.4 to about 7.5; an inorganic salt at a concentration of about 3 mM to about 100 mM; a membrane stabilizer at a concentration of about 3% to about 20% (wt.); a solution viscosity control reagent at a concentration of about 3% to about 30% (vol.); a water-soluble protein at a concentration of about 0.05% to 2.5% (wt.); and, a protease inhibitor at a concentration of about 0.05 mM to about 20 mM. More preferably, the composition has a pH from about 6.4 to about 7.5; an inorganic salt at a concentration of about 5 mM to about 25 mM or 50 mM; a membrane stabilizer at a concentration of about 5% to about 15% (wt.); a solution viscosity control reagent at a concentration of about 5% to about 20% (vol.); a water-soluble protein at a concentration of about 0.1% to 1.5% (wt.); and, a protease inhibitor at a concentration of about 0.05 mM to about 5 mM.

The particular embodiments of the inventive composition are described generally in terms of its components. An ideal standardized buffer should contain the following: a pH buffer, chemicals that determine ionic strength (inorganic salt), reagents that control water activity of biological membranes (membrane stabilizer), and generic proteins, which can help to pack biological membrane sheets (fragments) better within microspots and reduce the tendency of biological membranes to spread on a substrate surface during printing and post-printing processing. The buffer is made from a solution having commonly used pH control reagents, which may include Tris-HCl, HEPES, acetate, citrate, citrate-phosphate, phosphate, maleate, or succinate buffers. The inorganic salt may be selected from NaCl, KCl, CaCl₂, ZnCl₂, NiCl₂, MgCl₂, or MnCl₂.

To avoid either partial or complete dysfunction of receptors in the arrays due to a drastic change in the surrounding environment of the biomembranes during the printing process, post-printing manipulation, and storage, we propose using membrane stabilizers to improve array performance and extend shelf life. A membrane stabilizer or cryoprotectant may include monosaccarides (e.g., glucose, fructose), sugar alcohols (e.g., sorbitol, inositol), disaccharides (e.g., sucrose, trehalose, lactose, maltose), trisaccharides (e.g., raffinose), oligosaccharides (e.g., cycloinulohexaose), polysaccharides (e.g., ficoll, and dextran), polymers (e.g., poly-vinyl-pyrrolidone, polyethyleneglycol). Favored examples include disaccharides or oligosaccharides, or other molecules of similar water-activity controlling properties. The sugar molecules substitute for H₂O molecules. Typically water molecules help orient and support the biomembrane in an aqueous medium. Once a biological membrane is removed from the wet medium and is carefully dried, the lipid molecules in the biological membrane become disoriented and dysfunctional. The presence of disaccharides as substitutes for the water molecules prevents these problems associated with membrane desiccation.

Other reagents can be incorporated as part of the ink composition, including those that would change the viscosity of the ink for enhancing wettability for certain printing conditions. The viscosity control reagents may include glycerol, ethylene glycol, or dextran. The generic, water-soluable proteins may a wide range of protein which will not interfere with the binding of a target molecule with the probe receptors within a biological membrane microspot. Such proteins may include bovine serum albumin (BSA), Protein A, Protein G, or antibodies. The protease inhibitor may include EDTA, EGTA, 4-(2-aminoethyl)benzenesulfonyl fluoride (AEBSF), aprotinin, 1,10-phenanthroline, E-64, Antipain, Aprotinin, Benzamidine HCl, Bestatin, Chymostatin, E-aminocaproic acid, N-Ethylmaleimid, Leupeptin, pepstatin A, Phosphoramidon, Trypsin inhibitor, and any combination of these.

The medium possess a degree of stability that permits long-term storage of biological membranes in solution without excessive physical aggregation, chemical degradation, or physiological deterioration. Moreover, the medium enables superior adhesion to a functionalized substrate surface, as well as enhanced binding efficiency and specificity of the printed biological membranes.

According to certain embodiment for GPCR arrays, the pH buffer reagent used is preferably either Tris-HCl or HEPES, pH 7.0-7.5. The inorganic salt is preferably MgCl₂. Other types of salts, such as MnCl₂, can also be used according to the specific receptors. The membrane stabilizer is preferably either trehalose or sucrose. Glycerol or the like also can be included so as to achieve better viscosity in the receptor solution for sample pick-up and deposition by a pin. The generic protein is preferably a water-soluable protein such as bovine serum (BSA). It is known that BSA can form densely packed layer(s) on GAPS, thereby filling in the defects in the layer(s) of immobilized GPCR membrane fragments within a microspot. Introduction of BSA into the receptor solution can enhance the stability of microspots as well as reduce the chance of the biological membranes spreading during deposition. Most preferably, the receptor membrane preparations are re-suspended in a buffer containing: 50 mM Tris-HCl, pH 7.4, 10 mM MgCl₂, 0.5% BSA, ˜10% trehalose or sucrose, and ˜10% glycerol.

Sub-cellular fractionation techniques can partially separate and purify several important biological membranes, including the plasma and mitochondrial membranes, from many kinds of cells. Such biological membrane preparations generally have a varied distribution of lipid membrane fragments in different sizes. The empirical examples presented herein relate to GPCR arrays using cell membrane preparations, for example, by means of a contact pin-printing technology. Some properties of membrane preparations are important to their array performance. These properties include the relative homogeneity of a preparation, and the number of active receptor (Bₘₐₓ) and/or the total concentration of membrane-proteins within the preparation. A more homogeneous preparation having a higher Bₘₐₓ, gives rise to better array performance.

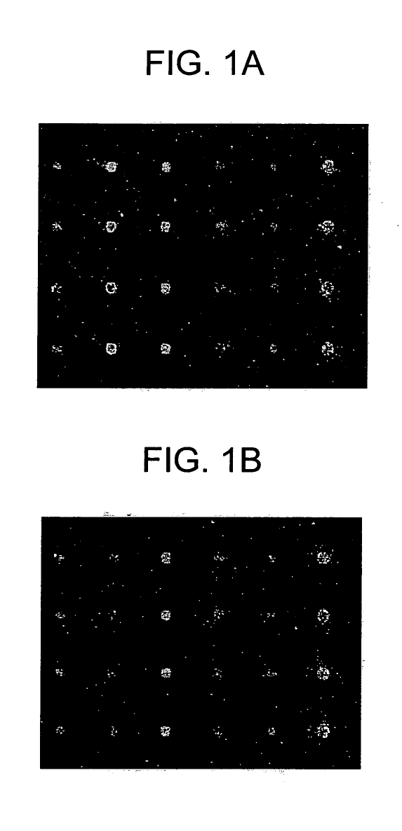

Advantages of the present invention are shown experimentally in the accompanying FIGS. 1-5. FIGS. 1A & 1B are false-color fluorescence image of a membrane microarray made using six different GPCR-membrane preparations. The images, in Cy5 to Cy3 ratio, are taken after the array is assayed with a binding buffered solution containing a mixture or cocktail solution of labeled ligands. The cocktail of solution contains 2 nM Cy3-B-telenzepine (a ligand for M1), 4 nM Bodipy-TMR-motilin 1-16 (a ligand for MOTR), 4 nM Cy5-Neurotensin 2-13 (a ligand for NTR1), 2 nM Cy5-nociceptin 1-13 (a ligand for ORL1), and 4 nM Cy5-naltrexone (a ligand for OP1). FIGS. 1A and 1B are, respectively, in the absence and presence of 1 μM telenzepin. Before array fabrication, all receptor membranes obtained from commercial vendors are re-formulated using a standardized ink containing: ˜20 mM Tris-HCl, pH 7.4, ˜10 mM MgCl2, ˜10% sucrose, 5% glycerol, 0.5% BSA, total membrane proteins in the range of 1 to 3 mg/ml. As shown in FIG. 1B, the presence of telenzepine, a specific antagonist of M1, specifically inhibits the binding of labeled ligand to M1 microspots, but not any other receptor microspots. The result of FIGS. 1A and 1B suggests that after reformuation with the present invention, even previously poor-performing, commercially-available membrane preparations can have enhanced performance.

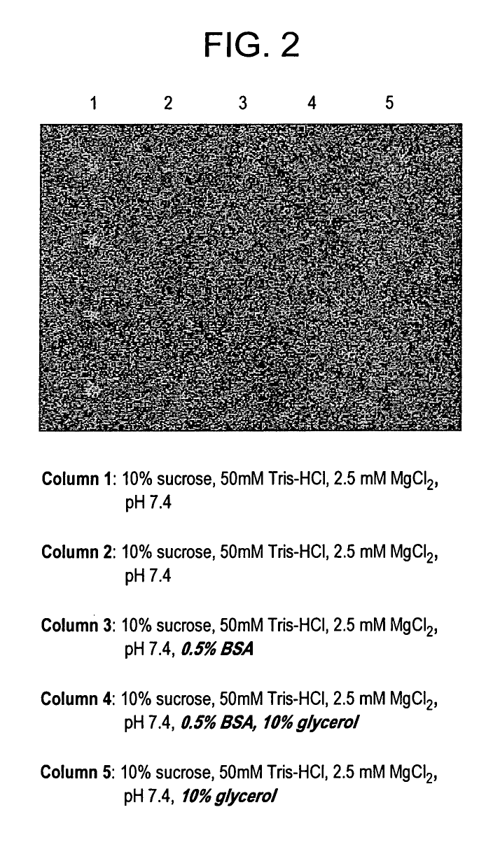

Under optimized conditions when using motilin receptors (MOTR) re-suspended in the inventive buffer medium, in FIG. 2, we observed that the membrane array had a lower autofluorescence of receptor microspots after being incubated with buffer alone. Except for column #1, where MOTR obtained from a commercial source (Perkin Elmer) is employed directly, all the receptors in the other four columns, #2-5, were diluted before printing by 1.5 fold to a final buffer concentration as indicated in the accompanying table. As one can see, for microspots corresponding to MOTR re-suspended in a buffer containing BSA and sucrose without glycerol, the array exhibits autofluorescence signals that are much lower.

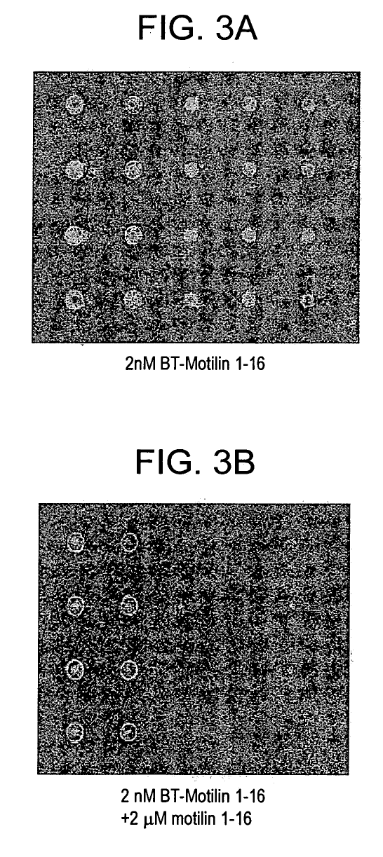

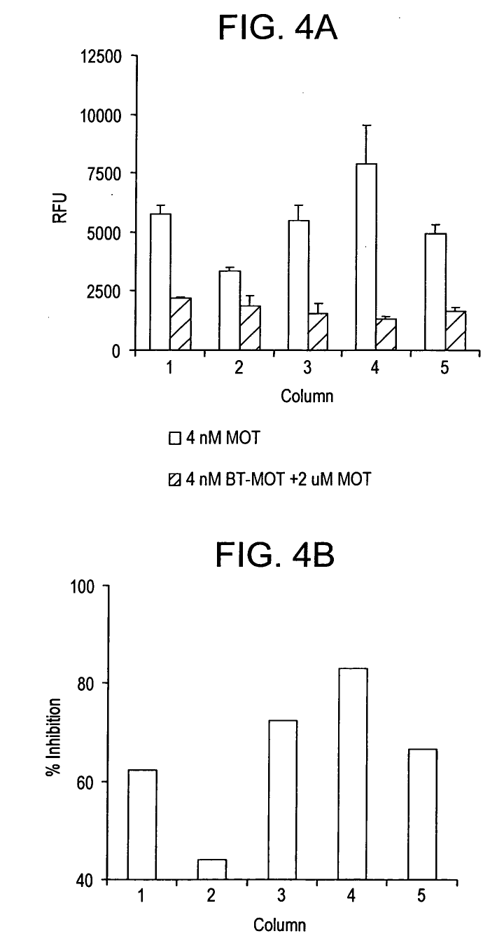

Also, note that the microspots have generally a better morphology, with a more confined area after bioassays (FIG. 3A, cols. 3 & 4) and less spreading of biological membranes after deposition; a higher total binding signals (FIGS. 3A & 4A); and a higher specificity of labeled motilin 1-16 binding to the MOTR in the arrays (FIGS. 3B, 4A & 4B). One can see a much higher total binding signals and specificity of the binding of BT-motilin 1-16 to the MOTR microarrays for MOTR re-suspended in a buffer containing BSA in the presence of glycerol and sucrose (col. 4).

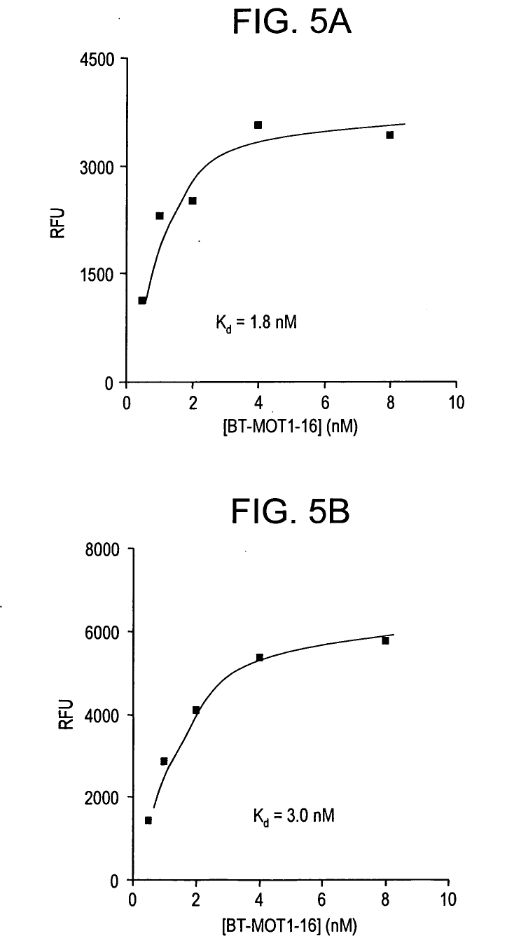

FIGS. 5A and 5B indicate a more precise and physiologically relevant binding constant. The plot analysis of FIG. 5A reveals that arrays of MOTR in the presence of BSA and sucrose gives rise to a K_{d} value of 1.8 nM. The plot analysis of FIG. 5B reveals that arrays of MOTR in the presence of BSA, sucrose, and glycerol gives rise to a K_{d} value of 3.0 nM. Both of these K_{d} values are close to that reported in the literature (˜2 nM).

When using the inventive composition, first provide a solution containing biological membranes. Add an undiluted volume of the buffered medium into the biological membrane containing solution until the final pH value and concentrations of each of the above mentioned components are within the prescribed ranges. Then, deposit the reformulated solution containing biological membrane onto a substrate surface at defined locations to form an array.

B. Confining & Stabilizing a Biological Membrane to a Predetermined Area

Biological-membrane arrays of the present invention are associated with a substrate surface. Several factors significantly affect the spot size, uniformity, stability, functionality and ligand-binding specificity of membrane-protein arrays (particularly for G-protein coupled receptor arrays) and their use in pharmacological ligand-binding assays. These factors include the printing conditions, printing ink compositions, surface chemistries, bioassay conditions, and receptor quality. Ideally, a biological membrane microarray should satisfy at least the following criteria: (1) The size of the microspots should be controllable thus enabling fabrication of an array with a desired density of spots; (2) The deposited membrane microspots should be confined in predetermined or designed locations before and after bioassays; (3) The printed microspots of biological membranes should be physically stable and resistant to removal from the surface over the course of a bioassay, which may include various preparation and handling treatments, such as incubation with a binding buffer, rinsing with different media, drying, or exposure of the microspots to air during handling; (4) Non specific binding of target molecules should be minimal; (5) The biological membranes on the surface should not spread uncontrollably or beyond predetermined tolerances during deposition and immobilization processes. The present invention successfully addresses these issues.

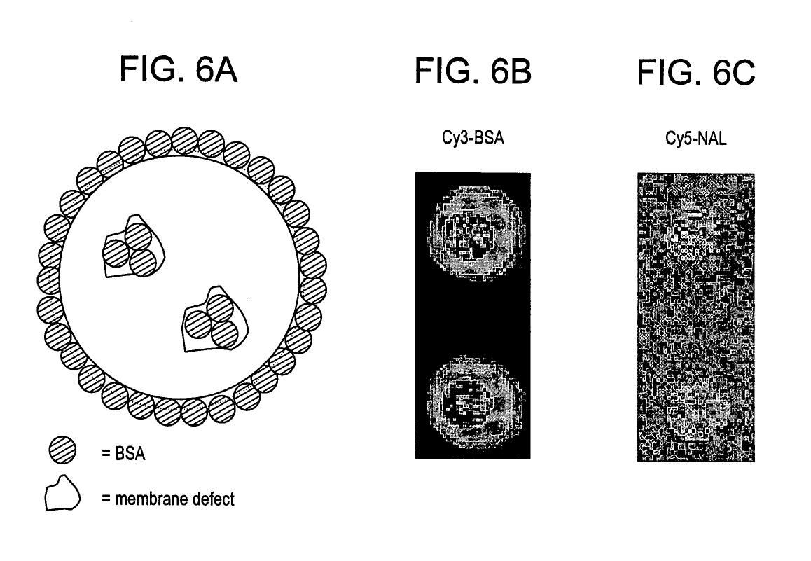

FIGS. 6A-C, respectively, depict in a schematic and fluorescent images the ability of the present ink compositions to confine and stabilize biological membranes within the defined area of a microspot. The ink composition contains any generic, water-soluble protein, such as bovine serum albumin (BSA), which will not interfere with the binding reaction of a target with the probe receptors. The biological membranes are suspended in a buffered solution containing the protein. After deposition onto a substrate surface, the water-soluble protein molecules tend to form a closely packed layer around the biological membranes, confining the membranes within a predetermined location and area of the microspot. Furthermore, the water-soluble protein molecules limit the ability of the membranes to spread during printing and post-printing process, and stabilize the membranes. Also, as occasion may require, the protein molecules may fill-in defect areas of the membrane microspot, thereby maintaining the integrity of the microspot.

Embodiments using BSA molecules can bind non-specifically to substrates such as bare glass, mica, gold, self-assembly monolayers of silanes and alkylthiols, and polymer-grafted surfaces. In some cases, the water-soluble proteins may form highly packed monolayers on the surface. These surface-bound proteins remain hydrated during the drying of the surfaces. This feature of the proteins is a great benefit, given that for certain kinds of substrates exposure to air can destroy the supported lipid membranes. Hence, according to the invention, these proteins are employed to form dense layers surrounding lipid and/or lipid/membrane protein spots. These protein layers significantly reduce the risk of biological membrane microarrays being exposed to air, and minimize damage to biological membranes caused by hydrodynamic forces when the membrane microarrays are withdrawn through water/air interfaces during rinsing and washing steps. Moreover, the proteins may serve to stabilize and anchor hydrophobic lipid bilayers by surrounding the edges of biological membrane microspots, which normally are susceptible to damage.

A further finding is that the water-soluble proteins, such as BSA, when introduced to a biological membrane preparation before deposition on a substrate, can limit the uncontrolled spreading of membrane deposits to generate microspots with desirable, closely-confined morphology and size. Previously, when BSA was incorporated and applied in a binding-reaction buffer solution, the spreading of the membranes could not be as easily controlled, since membranes tend to spread as soon as they are deposited on a surface during the printing and post-printing processes. Moreover, up until now, persons in the art believed generally that BSA would compete with a biological probe of interest to reduce the immobilization efficacy of biological membranes within a microspot. In contrast, our appreciation of the finding that BSA not only limits the spreading of membranes, but also does not interfere with immobilization within a microspot is shown to be greatly beneficial to the manufacture of membrane microarrays.

C. Membrane Protein Arrays with Enhanced Assay Performance & Prolonged Shelf-Life

As noted previously, certain biological membrane microarrays can be produced, stored and used, not only in an aqueous environment, but also in an environment exposed to air under ambient or controlled humidity conditions. The fabrication, post-deposition and storage processes for GPCR arrays can involve drastic changes in environment surrounding the biological membranes. These changes could cause a number of effects on the biological function of the embedded receptors, such as partial dysfunction and low specificity of ligand binding to the receptors in the arrays, or shortened shelf-life.

According to another aspect of the present invention provides a way to prevent deterioration of lipid membranes when they are exposed to ambient conditions. Specifically, the method involves using disaccharide or oligosaccharide molecules (e.g., sucrose or trehalose), or similar compounds, to prevent fusion and aggregation in lipid membranes in solution before printing, and to maintain the integrity of biological membranes against dehydration after printing and during storage. The sugar molecules are used as membrane stabilizer, which regulate water activities around lipid head groups, thereby stabilizing the lipid membrane when exposed to ambient conditions. In particular, the present methods helps lipid molecules in membranes retain their relative orientations.

When incorporated in a fabrication process, the present method step can prevent fusion and aggregation between lipid membranes in solution during the solution preparation and array fabrication processes; and hence, can provide a membrane or membrane-protein microarray of superior assay performance and prolonged shelf-life relative to similar types of conventional arrays. After deposition of the membranes on an array substrate, the present method allows the membranes to maintain their functional integrity and orientation against dehydration during printing, post-printing processes and storage under ambient conditions. In particular, the present method can help lipid molecules in membranes retain their relative orientations, and reduce the possibility of dysfunction of membrane proteins embedded in membranes, thus enhancing assay performance.

The organization of water at a lipid/membrane interface determines important functional properties of biological membranes. Trehalose and other oligosaccarides have been reported to be efficient crytoprotectants, due to the replacement of water at the membrane structure. (Luzardo, M. C., Amalfa, F., Nunez, A. M., Diaz, S., Lopez, A. C. B., Disalvo, E. A., “Effect of Trehalose and Sucrose on the Hydration and Dipole Potential of Lipid Bilayers,” Biophys. J. 78, 2452-2458 (2000).) In particular, trehalose is very effective at replacing water molecules associated with polar lipid-head groups and stabilizing biological membranes when the membrane bilayers are dried or frozen under drastic conditions. For these reasons, trehalose is commonly used by the pharmaceutical industry to increase the stability of liposome systems. This action of trehalose has been attributed to its hydrogen bonding capacity, its glass characteristics, its ability to modify the lipid salvation layer, and its large hydrated volume when compared to those of other carbohydrates (e.g. 2.5 times that of sucrose). (Bardos-Nagy, I., Galantai, R., Laberge, M., Fidy, J. “Effect of Trehalose on the Nonnond Associative Interactions Between Small Unilamellar Vesicles and Human Serum Albumin and on the Aging Process,” Langmuir 19, 146-153 (2003).) This unique property of trehalose as biomembrane stabilizer against drying, however, has not been applied to biological membrane microarrays in general, nor to GPCR microarrays specifically.

The stabilizing effect of disaccharides on membranes reduces the possibility of dysfunction of membrane proteins embedded in membranes, thereby enhancing assay performance (e.g., ligand binding specificity) and permitting one to storage the printed GPCR array under less stringent conditions and for longer periods than done ordinarily. In certain embodiments, the method comprises applying disaccharides at certain concentrations to a biological membrane suspension before depositing onto a substrate surface. The concentrations of disaccharides are preferably about 1% to 25% (wt.), or more preferably about 5-10% in an aqueous buffered solution.

According to an embodiment of the method, one can suspend biological membranes in a buffer containing a certain concentration of the membrane stabilizer. For instance, biological membranes including GPCR membrane preps can be suspended in a buffer containing ˜10% trehalose. The biological membranes suspended in trehalose-containing buffer are applied to fabricate arrays, including using contact pin printing technology. In another example, different amounts of trehalose were added to human β-adrengeric receptor subtype 1 (β1) membrane preps and deposited on GAPS slides using quill pin contact printing technology to create β-arrays. After deposition, the β-arrays were incubated under high humidity chamber at room temperature for one hour, followed by stored at room temperature under vacuum condition for certain time. A standard binding assay was used to evaluate the array performance and ligand binding specificity.

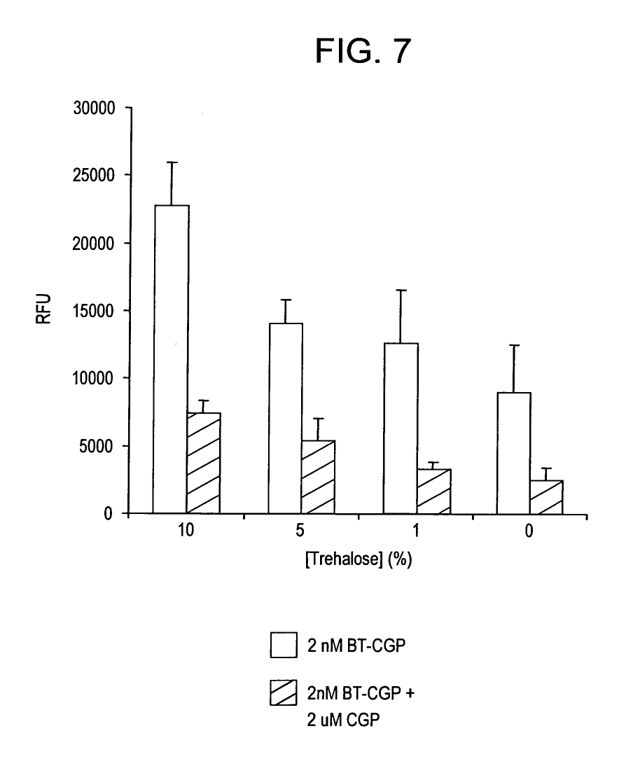

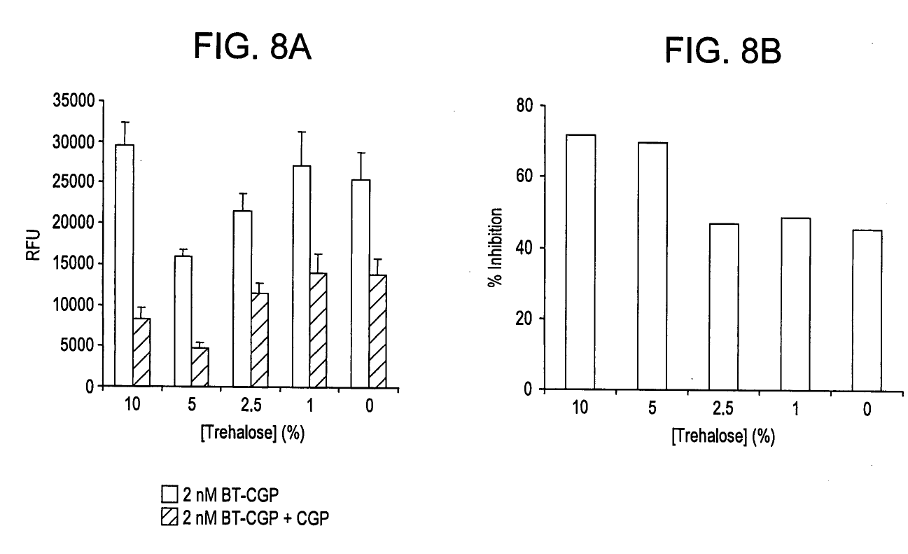

According to another feature of the method, the invention affords researchers the ability to store stable arrays under a much less stringent condition than usual. For example, a fabricated biological membrane array may be stored at room temperature under a nitrogen atmosphere or vacuum condition. Data from the example, above, are presented in FIGS. 7 and 8. The results reveal that the presence of trehalose in the membrane preparations enhance assay performance of GPCR arrays in terms of total binding signals and ligand binding specificity. Counter to standard storage conditions, which typically are at about 4° C. or lower temperatures, the less stringent storage conditions employed in the present method can reduce the complexity of array handing and shipment to end users. Moreover, GPCR arrays remain fully functional even after being stored under much less stringent conditions (e.g., at room temperature under a vacuum), suggesting that the presence of trehalose or sucrose improves the shelf-life of GPCR arrays.

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A method for fabricating a biological membrane array, the method comprising: a) reformulating a biological membrane suspension using a medium comprising a soluble protein; and b) depositing the reformulated biological membrane suspension onto a solid support to form a microarray comprising microspots, such that the soluble protein forms a layer surrounding the biological membrane microspots.

27. The method according to claim 26, wherein said depositing step further comprises: a) immersing a tip of a pin into the reformulated biological membrane suspension; b) removing said tip from the reformulated biological membrane suspension with the reformulated biological membrane suspension adhered to the pin tip; and c) transferring the reformulated biological membrane suspension to said solid support.

28. The method according to claim 26, wherein said depositing step may be repeated a plurality of times to provide one or more arrays of biological membranes.

29. The method according to claim 27, wherein a typographic pin printing technique is employed.

30. A method for stabilizing a biological membrane array, the method comprising: a) reformulating a biological membrane suspension using a medium comprising a membrane stabilizer; and b) depositing the reformulated biological membrane suspension onto a substrate surface to form a microarray comprising microspots, wherein the membrane stabilizer replaces water molecules associated with the biological membrane structure within the microspot.

31. (canceled)

32. A method for making a biological membrane array, the method comprising: a) providing a solution containing biological membranes; b) introducing both a soluble protein and a membrane stabilizer into the solution containing biological membranes and c) depositing said reformulated solution containing biological membrane onto a substrate surface at defined locations to form an array.

33. The method according to claim 26, wherein the biological membrane microspots comprise binding functional molecules.

34. The method according to claim 33, wherein the binding functional molecules are selected from the group consisting of a ganglioside, phosphatidylinositol phosphate, a sphingolipid and a membrane-protein.

35. The method according to claim 34, wherein the membrane-protein is selected from the group consisting of a GPCR, a ligand-gated ion channel receptor, a tyrosine kinase receptor, a serine/threonine kinase receptor and a guanylate cyclase receptor.

36. The method according to claim 26, wherein the reformulated biological membrane suspension comprises a supported lipid bilayer membrane, a bilayer vesicle, a lipid micelle, at least a partially free-suspended lipid membrane, or a lipid membrane in a nano-channel of a substrate.

37. The method according to claim 26, wherein the soluble protein is characterized as a protein which will not interfere with the binding of a target molecule with the probe receptors within the biological membrane microspot.

38. The method according to claim 37, wherein the soluble protein is selected from the group consisting of bovine serum albumin, Protein A, Protein G and an antibody.

39. The method according to claim 30, wherein the membrane stabilizer is a chemical, polymer, biochemical or biological wherein physical damage of membranes during freezing, drying, or exposing to ambient conditions is minimized.

40. The method according to claim 39, wherein the membrane stabilizer is selected from the group consisting of a monosaccaride, a sugar alcohol, a disaccharide, a trisaccharide, an oligosaccharide and a polysaccharide.

41. The method according to claim 40, wherein the membrane stabilizer is selected from the group consisting of glucose, fructose, sorbitol, inositol, sucrose, trehalose, lactose and maltose.

42. The method according to claim 32, wherein the solution containing biological membranes comprises a) a pH buffer reagent having a pH from 5.8 to 7.8; b) an inorganic salt at a concentration of 1 mM to 500 mM; c) the membrane stabilizer at a concentration of 1% to 30% (wt./wt.); d) a glycerol at a concentration of 1% to 55% (vol./vol.); and e) the soluble protein at a concentration of 0.01% to 3% (wt./wt.).
